(19) United States
(12) Patent Application Publication
(10) Pub. No.: US 2023/0146699 A1
(43) Pub. Date: May 11, 2023

(54) EDTA System for Reducing Browning in Mycohide Materials

(71) Applicant: MycoWorks, Inc., Emeryville, CA (US)

(72) Inventors: Ritu Bansal-Mutalik, Albany, CA (US); Mahla Zabet, Santa Monica, CA (US)

(21) Appl. No.: 17/982,956

(22) Filed: Nov. 8, 2022

Related U.S. Application Data
(60) Provisional application No. 63/277,126, filed on Nov. 8, 2021.

(51) Int. Cl. C12N 1/04

(52) U.S. Cl. CPC C12N 1/04

(57) ABSTRACT

A system and methods for reducing browning in fungal materials and objects therefrom, and in particular to a method for reducing browning in processed fungal materials. Provided are particular methods for reducing browning in fungal materials via application of a chelating agents, heat, a combination of chelating agents and heat, and various other processes described herein. Reduction of browning is applied to mycelium sheets in various wet processing stages including fermentation, plasticization, and in temperature treated samples. Reduced browning allows for control of the appearance of fungal biopolymers while also impacting mechanical properties such as tensile strength, tear strength, and abrasion resistance.

### PRIORITY

The present disclosure claims priority to provisional patent application 63/277126 filed Nov. 8, 2021, which is incorporated herein as if set out in full.

### TECHNICAL FIELD

The present disclosure relates generally to methods for reducing browning in mycohide materials, and in particular to a method for reducing browning in fungal materials using varied application of a chelating agent, heat, or a combination of chelating agents and heat. A sequence of steps for reducing browning in fungal materials may supplement standard mycelium plasticization and tanning steps known in the art.

### BACKGROUND

Mushrooms are attractive resources for novel enzymes, bioactive compounds, and mechanical functional compounds. However, mushrooms spontaneously form brown pigments during food processing as well as extraction procedures. Fungi, including yeasts, molds, and mushrooms, are attractive resources for biotechnological applications. Of these organisms, many mushroom species have historically been used for food or medicinal purposes. In the past decade, there has been growing research interest in the use of mushroom species for the formation of building materials and mycelium products such as mycelium leather substitutes. The typical mushroom fruiting body is usually white initially, but, when processed, the mushroom sometimes develops brown to black pigments. Indeed, in standard laboratory purification processes for both mushroom proteins and bioactive compounds, solution extracted from the typical mushroom lysate is pale yellow, and the extract darkens over time during these processes.

The oxidation reaction of polyphenols and other oxidizable substances can potentially be prevented by antioxidant compounds in the mushroom fruiting body. However, oxidizable and aromatic-ring-containing substances in the mushroom fruiting-body extract can be exposed, resulting in their oxidization under aerobic conditions. Similar to well-known plant substances, a number of aromatic compounds, including hericines, erinacines, alkalonides, lactones, and their derivatives, can presumably be converted to pigment-forming molecules. These mushroom metabolite substances are possibly oxidized products that are formed by polyphenol oxidases (PPOs) in the mushroom fruiting body.

PPOs, including tyrosinase and laccase, are enzymes well-known for their activities related to post-harvest browning pigments in agricultural products. Although these pigment compounds, such as certain secondary metabolites, might be useful for enhancing bioactive compounds in fermentation processes or for helping to preserve protein in forage crops, the browning reactions are generally viewed negatively in mycelium products such as mycelium leather substitutes. The dark color reaction occurs via PPOs, which can be induced by wounding or pathogen attacks as part of the defense response in plants and fungi. Indeed, it has been shown that PPOs play such a role in the white button mushroom (Agaricus bisporus) (See Soler-Rivas et. al. Activation of tyrosinase in Agaricus bisporus strains following infection by Pseudomonas tolaasii or treatment with a tolaasin-containing preparation. Mycol. Res. 1997, 101, 375-382.) Nonetheless, the exact kinds of substances in the mushroom that are converted to brown products by some PPOs are unknown in mushroom species.

The present inventors have proposed and documented that chelating agents may be used to reducing browning via interference with the endogenous enzymatic reaction of the PPO family, reducing the occurrence of this pigmentation. PPO family members are known metalloenzymes, employing metal ions at their active sites. Chelating agents such as EDTA and EGTA may hypothetically be employed to sequester metals in proximity or in direct contact with processed mycelium materials, thereby preventing their use by PPO enzymes. Specifically, chelating substances may be used to inhibit PPO activity because they can form complexes with Cu(II) present in PPO or react with their substrates, thereby suppressing enzymatic browning.

Notably, synthetic chelates such as ethylenediaminetetraacetic acid (“EDTA”) and egtazic acid (“EGTA”) are commonly used to sequester metal ions in aqueous solution. In the textile industry, they prevent metal ion impurities from modifying colors of dyed products. In the pulp and paper industry, EDTA inhibits the ability of metal ions, especially Mn²⁺, from catalyzing the disproportion of hydrogen peroxide, which is used in chlorine-free bleaching. In a similar manner, EDTA is added to some food as a preservative or stabilizer to prevent catalytic oxidative discoloration, which is catalyzed by metal ions. In soft drinks containing ascorbic acid and sodium benzoate, EDTA mitigates formation of benzene (a carcinogen).

Generally speaking, the properties and applications of fungal materials are strongly linked to their morphology, structure and size. In some cases, fungal materials may form a composite with other materials such as cotton textiles and/or chitin nanowhiskers. Such composites can be used for various applications and are widely utilized in textiles, packaging and building materials. The properties of fungal materials may be controlled by various methods, including plasticization. Plasticization allows for control of several important parameters including tensile strength, tear strength, abrasion resistance, in addition to various chemical properties such as dye fixation and optical homogeneity. Plasticization may also help to optimized how putrescible or stabilized a given fungal material may be in a given end product. At a microscopic scale, distinct chemical bonding arrangements may be available for plasticization of fungal materials when compared to collagen or similar materials (animal leathers are composed of collagen, which is an organic, fibrous material). Fungal materials, on the other hand, are primarily comprised of chitin. Chitin is a molecularly-distinct organic fiber material with a distinct make-up of hydroxyl versus amine groups available for chemical plasticization.

As applied to reduced browning mycohide materials, a reduced browning fungal material may be comprised of natural or modified fungal proteins, carbohydrates, and nucleic acids. Fungal materials also generally comprise a network of interlocking branched hollow tubes called hyphae. As described above, hyphae contain a unique molecular compound called chitin. Chitin is also the main constituent in the shells of crustaceans and is the most abundant naturally occurring biopolymer other than cellulose. Chitosan is derived from chitin and can be formed by deacetylation of chitin. Chitosan is commercially available in a wide variety of molecular weights (e.g., 10-1,000 kDa) and usually has a degree of deacetylation ranging between 70% and 90%. Chitosan is used for a wide variety of purposes including plant care, cosmetics additives, food and nutrition supplements and medical care.

Filamentous fungi have the natural tendency to join together smaller pieces of branching, colonial hyphae into a larger constituent whole, assembling and weaving strands and sheets of tissues called mycelium. Mycelium can adhere to, and possibly engulf, any other materials it comes in contact with through the extension of hyphae that use neighbor sensing and searching functions as guidance in their exploration into space beyond sources of nutritional sustenance. Like cement and plaster, fungal tissue will bind, harden and set into a variety of solidified configurations through the natural biological functions of mycelial growth and self-adhesion. In some instances, fungal tissues can quickly be amplified to a large volume if provided with the appropriate living conditions. These conditions include the nutrients that might be available to the organism, the possible gas gradients within the growth environment and the humidity, light, and temperatures the organism might be exposed to as it takes form. Fungi are very sensitive to their surroundings, and by altering subtle factors it is possible to prompt their tissue to express a range of variably determined physical characteristics.

Fungi are very sensitive to chemicals present in their environment, and have the ability to alter the directions and vigor of growth of expanding hyphae as demonstrated through chemotaxic avoidance or attraction. Fungi are also very sensitive to other stimuli in their environment, and have the ability to alter directions and vigor of growth of expanding hyphae in response to gravitropic, thermotropic, thigmotropic, phototropic, and hydrotropic stimuli. A substrate colonized with fungal hyphae, if provided adequate enclosure and environmental controls, will in a matter of one to three days generate a layer of fungal hyphae growing from the top of said substrate that will expand into space as a layer in a fuzzy and undifferentiated manner. This undifferentiated layer of hyphae, if left to continue growing, will soon advance in development and differentiate into specialized tissues determined to become fruit bodies or other sporocarp-producing structures. Background material discussed above relies on various authorities including Michael Sullivan and Seonghun Kim (See Sullivan, Michael L. Beyond brown: polyphenol oxidases of plant specialized metabolism, Frontiers in Plant Science, January 2015; also See Kim, Seonghun, Foods 2020, 9(7), 951).

In some instances, fungus-based materials and composites can be propagated on readily available agricultural waste, using principles and techniques that are well established with regard to growing filamentous fungi for human consumption and industry. Notably, whereas cellulosic materials have been shown to be physically altered through plasticization, fungal materials have not been successfully reduced browning with glycerin and the methods described herein in order to achieve homogenous optical densities. Under optimized conditions, fungal composites may be altered through plasticization in order to exhibit equivalent or improved properties and characteristics as compared to animal skins and similar materials.

### SUMMARY OF THE INVENTION

To minimize the limitations found in the existing systems and methods, and to minimize other limitations that will be apparent upon the reading of this specification, the present invention includes methods for reducing browning in processed fungal materials, and in particular to a method for reducing browning in fungal materials using varied application of a chelating agents, heat, or a combination of chelating agents and heat. A sequence of steps for reducing browning in fungal materials may supplement standard mycelium plasticization and tanning steps known in the art.

Certain embodiments of the present invention provide methods for reducing browning in a fungal material that was originally comprised predominately of organic fungal tissues. The resultant material is a flexible, optically homogenous, high-density polymer that takes on a reduced browning or “whiter” appearance relative to mycelium browned by standard fat liquoring and/or drying processes. As is known in the art, reducing browning allows for control of the appearance of fungal biopolymers, though it may also impact mechanical properties such as tensile strength, tear strength, abrasion resistance and other chemical properties such as dye fixation.

A first objective of the present invention is to provide a structure comprised of reduced browning fungal materials and their composites wherein the mechanical and chemical properties of the fungal materials and their composites are well-controlled.

A second objective of the present invention is to successfully modify browning in fungal materials such that the finished product appears akin to an animal leather, common industrialized animal skin, or the like. This may be achieved by application of chelating agents at wet processing steps during fermentation, harvesting, plasticization, or tanning.

It is another objective of the present invention to provide a method for producing a fungal materials and structures of variable shape, thickness, density, flexibility and treated with different temperatures at different durations for industrial applications.

Yet another objective of the invention is to provide a method for reducing browning in wet fungal sheets, plasticized sheets, and the like in order to enhance desired characteristics such as improved flexibility and tensile strength.

Yet another object of the invention is to provide a method for reduced browning that facilitates deactivation of fungal growth.

Yet another object of the invention is to facilitate the post processing of reduced browning fungal materials while mitigating environmental impacts of processing.

Yet another object of this invention is to provide a reduced browning fungal material for use in functional products.

Yet another object of the invention is to provide a material that can act as an analog to synthetic plastic materials, foams, and animal skins.

These and other advantages and features of the present invention are described with specificity so as to make the present invention understandable to one of ordinary skill in the art. The following detailed description together with accompanying figures will provide a better understanding of the nature and advantages of the present invention.

### BRIEF DESCRIPTION OF THE DRAWINGS

In order to enhance clarity and improve understanding of the various elements and embodiments of the invention, elements in the figures have not necessarily been drawn to scale. Furthermore, elements that are known to be common and well understood to those in the industry are not depicted in order to provide a clear view of the various embodiments of the invention. Thus, the drawings are generalized in form in the interest of clarity and concision.

FIG. 1A shows the front aspect of a wet felt sheet of mycohide prior to tanning;

FIG. 1B shows the front aspect of a wet felt sheet of mycohide after tanning;

FIG. 1C shows the back aspect of a wet felt sheet of mycohide after tanning;

FIG. 2A shows the front aspect of a plasticized felt sheet before tanning;

FIG. 2B shows the front aspect of a plasticized felt sheet after tanning;

FIG. 2C shows the back aspect of a plasticized felt sheet after tanning;

FIG. 3A shows the front aspect of a wet cotton sheet of mycohide prior to tanning;

FIG. 3B shows the front aspect of a wet cotton sheet of mycohide after tanning;

FIG. 3C shows the back aspect of a wet cotton sheet of mycohide after tanning;

FIG. 4A shows the front aspect of a plasticized cotton sheet before tanning;

FIG. 4B shows the front aspect of a plasticized cotton sheet after tanning;

FIG. 4C shows the back aspect of a plasticized cotton sheet after tanning;

FIG. 5A shows the impact of 1 mM EDTA on reducing surface browning;

FIG. 5B shows the impact of 5 mM EDTA on reducing surface browning;

FIG. 6A shows the impact of 1 mM EGTA on reducing surface browning;

FIG. 6B shows the impact of 5 mM EGTA on reducing surface browning;

FIG. 7 shows heat treated (no EDTA) crusts after fatliquoring (wet state), with numbering on each piece representing the time period in hours of heat treatment;

FIG. 8 illustrates heat treated (no EDTA) after fatliquoring (dry state before exposing to sunlight);

FIG. 9 shows crusts after fatliquoring (wet state);

FIG. 10 illustrates crusts after fatliquoring (dry state);

FIG. 11 shows a first example of the effect of lower temperature ranges on discoloration/browning of a plasticized sheet;

FIG. 12 illustrates shows a second example of the effect of lower temperature ranges on discoloration/browning of a plasticized sheet;

FIG. 13 shows the effect of EDTA application at 1 mM and 5 mM EDTA in the presence and absence of heat;

FIG. 14 shows a schematic of an exemplar plasticization process using glycerin solution on four different harvested mycelium sheets, wherein EDTA is applied at different points in the plasticization process;

FIG. 15 shows an exemplar design for a mycelium tanning process following plasticization;

FIG. 16 illustrates harvested wet and plasticized sheets in the presence of EDTA, heat, or both EDTA and heat;

FIG. 17 shows dried plasticized sheets after treatment of wet harvested sheets with EDTA, heat, or both EDTA and heat;

FIG. 18A shows that the application of heat in a first example, with or without EDTA, does not have a consistent significant added effect in terms of browning reduction;

FIG. 18B shows that the application of heat in a second example (Sheet ID: 5180-2166), with or without EDTA, does not have a consistent significant added effect in terms of browning reduction;

FIG. 19 shows a schematic of an exemplar process pipeline including various potential steps wherein EDTA may be applied;

FIG. 20A shows the results from a first exemplar set/sheet wherein EDTA is added at different process steps and under varied fat liquor solution conditions; and

FIG. 20B shows the results from a second exemplar set/sheet wherein EDTA is added at different process steps and under varied fat liquor solution conditions.

### DETAILED DESCRIPTION OF THE INVENTION

In the following discussion that addresses a number of embodiments and applications of the present invention, reference is made to the accompanying drawings that form a part hereof, and in which is shown by way of illustration specific embodiments in which the invention may be practiced. It is to be understood that other embodiments may be utilized and changes may be made without departing from the scope of the present invention.

Various inventive features are described below that can each be used independently of one another or in combination with other features. However, any single inventive feature may not address any of the problems discussed above or only address one of the problems discussed above. Further, one or more of the problems discussed above may not be fully addressed by any of the features described below. As used herein, the singular forms “a”, “an”, and “the” include plural referents unless the context clearly dictates otherwise. “And” as used herein is interchangeably used with “or” unless expressly stated otherwise. As used herein, the term “about” means +/−5% of the recited parameter. All embodiments of any aspect of the invention can be used in combination, unless the context clearly dictates otherwise.

Unless the context clearly requires otherwise, throughout the description and the claims, the words “comprise”, “comprising”, and the like are to be construed in an inclusive sense as opposed to an exclusive or exhaustive sense; that is to say, in the sense of “including, but not limited to”. Words using the singular or plural number also include the plural and singular number, respectively. Additionally, the words “herein,” “wherein”, “whereas”, “above,” and “below” and words of similar import, when used in this application, shall refer to this application as a whole and not to any particular portions of the application. The description of embodiments of the disclosure is not intended to be exhaustive or to limit the disclosure to the precise form disclosed. While the specific embodiments of, and examples for, the disclosure are described herein for illustrative purposes, various equivalent modifications are possible within the scope of the disclosure, as those skilled in the relevant art will recognize.

As noted above, chelating agents like EDTA have been used previously in foods, pharmaceuticals, biomaterials and other industrial applications. Here, EDTA is employed in a unique manner in that it is being used for mycelial based biomaterial/bioleather browning reduction to facilitate the production of leather substitutes. Applications of the reduced browning methods described herein also include building material applications. In addition to the application of chelating compounds, other methods of browning reduction contemplated by the present inventors include: 1) heat (high temp >50 deg C, thereby inducing inhibition of all enzymes including PPOs that cause browning in mycelia), 2) high pH extractions (proteins including PPO enzymes are extracted out with alkaline solutions), 3) low pH treatment (low pH precipitates some of the PPO enzymes), and 4) application of antioxidant agents (reducing the antioxidant activity of PPOs).

In a preferred embodiment, the invention involves chelating agents like EDTA (“Ethylenediaminetetraacetic acid”) and others as possible agents used to reduce browning in mycelial biomaterials for various applications including fine mycelium. Chelating agents also render the mycelial material a lighter shade and/or form a white background, which improves aesthetics while enabling dyeing of the mycelial material with any light shade of color. Notably, EDTA treatment may be beneficially applied at any mycelium processing stem wherein the mycelium is saturated or moistened. The reduced browning methods described herein may be applied to any of the steps in the mycelium processing pipeline wherein the mycelia are in a wet or moist state, including during growth, post-harvest during plasticization, or during downstream and/or tanning steps.

As described above, the present invention further relates to a composition containing a dipping or soaking solution useful for at least one mycohide crust or sheet to be dipped or soaked therein, wherein the dipping or soaking solution contains at least one metalloproteinase inhibitor. In other embodiments, the metalloproteinase inhibitor is accompanied or followed by treatment in a solution of 15% ALE fatliquor+EDTA (either 1 mM or 5 mM EDTA). The metalloproteinase inhibitor can be a chelator, such as a chelator of a divalent metal ion (i.e., preferably EDTA or EGTA). As a non-limiting alternate example, the metalloproteinase inhibitor may be an aminocarboxylic acid or a polyaminocarboxylic acid or a salt thereof.

As a non-limiting example of a preferred embodiment, the metalloproteinase inhibitor may be ethylenediaminetetraacetic acid (EDTA) or a salt thereof, such as, for example, ethylenediaminetetraacetic acid disodium salt (Na2EDTA or disodium EDTA), ethylenediaminetetraacetic acid trisodium salt (Na3EDTA or trisodium EDTA), ethylenediaminetetraacetic acid tetrasodium salt (Na4EDTA or tetrasodium EDTA), ethylenediaminetetraacetic acid dipotassium salt (K2EDTA), ethylenediaminetetraacetic acid tripotassium salt (K3EDTA), ethylenediaminetetraacetic acid ammonium salt (NH4EDTA), or ethylenediaminetetraacetic acid diammonium salt ((NH4)2EDTA). As a non-limiting example, the metalloproteinase inhibitors may be ethylene glycol-bis((3-aminoethylether)-N,N,N′N′-tetraacetic acid (EGTA) or a salt thereof, such as, for example, the tetrasodium salt, Na4EGTA. Other non-limiting examples of metalloproteinase inhibitors include S,S′-ethylenediamine disuccinic acid (EDDS), 1,2-diaminocyclohexene-N,N,N′,N′-tetraacetic acid (CDTA) and N-(2-hydroxyethyl)ethylenediamine-N,N′-triacetic acid (HEEDTA).

Still other non-limiting examples of metalloproteinase inhibitors include methylglycinediacetic acid (MGDA), N,N-bis(carboxymethyl)glutamate (GLUDA), ortho-phenanthroline, 8-hydroxyquinoline, and phosphonic acid derivatives such as amino-tris methylene phosphonic acid, e.g., sold by Buckman Laboratories under the tradename “Phos 2”, diethylene triamine pentamethylene phosphonic acid, e.g., sold by Buckman Laboratories under the tradename “Busperse 254”, 2-phosphono-1,2,4-butanetricarboxylic acid, e.g., sold by Buckman Laboratories under the tradename “Phos 9”, hydroxyethylidene-diphosphonic acid, e.g., sold by Buckman Laboratories under the tradename “Phos 6”, or a blend of 2-methylpentanediamine tetrakis (methylene phosphonic acid) and 1,2, diaminocyclohexanetetrakis (methylene phosphonic acid), e.g., sold by Buckman Laboratories under the tradename “BPS 319.” Still other examples of metalloproteinase inhibitors include citric acid and salts of citric acid, gluconic acid, and salts of gluconic acid, cysteine, iodoacetic acid and sodium iodoacetate. Mixtures of any of the compounds named herein may also be used.

In some embodiments, the amount of the metalloproteinase inhibitor contained in the dipping or soaking solution is not critical and may be any amount effective to reduce browning in the treated mycohide sheets. As an example, the amount of the metalloproteinase inhibitor in the dipping or soaking solution can be from about 0.00001% to about 18%, from about 0.0001% to about 5%, or alternatively from about 0.001% to about 2% by weight based on the weight of the mycohide sheet contained in the composition. Alternatively, the amount of the metalloproteinase inhibitor in the dipping or soaking solution can be from about 0.00001% to about 10%, preferably from about 0.0001% to about 5% and most preferably from about 0.001% to about 2% by weight based on the dipping or soaking solution. The dipping or soaking solution can be any solution, such as, for example, an aqueous solution, that is used to treat mycohide sheets, including, but not limited to cleaning, chilling, curing, and/or solutions for softening or hydrating a mycohide sheet before or after plasticization and tanning.

In some embodiments, EDTA solution above 0.5 mM or above is used to treat mycelial material. Other chelating agents like EGTA (β-aminoethyl ether)-N,N,N′,N′-tetraacetic acid), DTPA (diethylenetriaminepentaacetic acid) and GLDA (Dissolvine® GL is glutamic acid diacetic acid, tetra sodium salt) can be used to reduce browning in mycelial biomaterials as well. In a preferred embodiment, EDTA can be used in any form, including the non-salted form (i.e., Sigma Aldrich Product Number: E9884, Index-No.: 607-429-00-8, CAS-No.: 60-00-4). Preparation conditions for chelating agents vary. For example, the above-referenced EDTA (Product Number E9844) is prepared by dissolving in NaOH solution (i.e., 12 mM NaOH).

In some embodiments, the mycelium is heated at 50-70 deg C and EDTA treated. In another embodiment, under alternate conditions, heating is not implemented as EDTA alone provides a better equivalent impact. Notably, heating at scale is also a challenge, thus motivating the EDTA-only approach. As described below, optimal conditions were arrived at by first optimizing concentrations of EDTA and another chelating agent EGTA. A concentration of 5 mM EDTA was revealed to be the optimum concentration to achieve browning reduction. In some embodiments, non-salted EDTA concentrations can range from 2 mM to 12 mM (i.e., its solubility limit in alkaline solution). In a preferred embodiment, 5 mM is the ideal concentration to use for browning reduction, however lower EDTA concentrations will have an impact. For example, salted EDTA is used at concentrations exceeding 5 mM, including for example 6 mM, 8 mM, 10 mM, and 15 mM. Notably, chelating agents can be used in combination with heat or any other treatment that can reduce browning further.

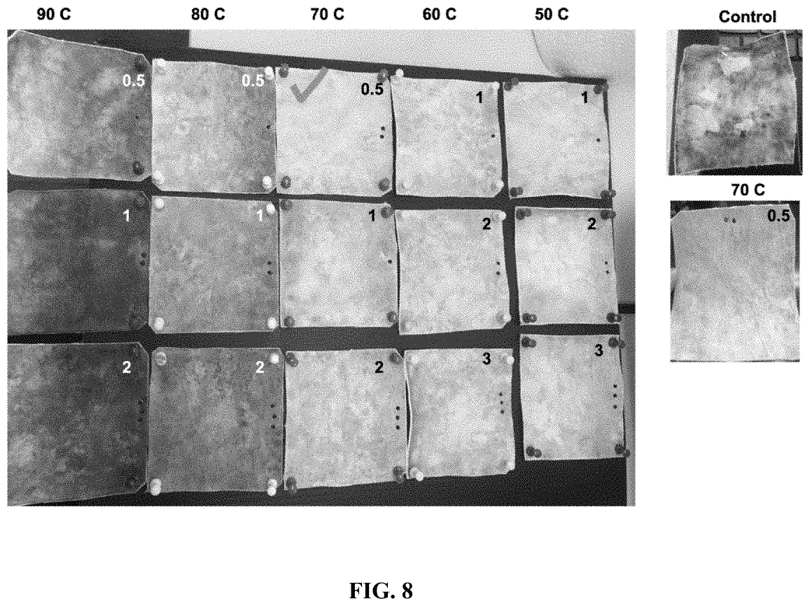

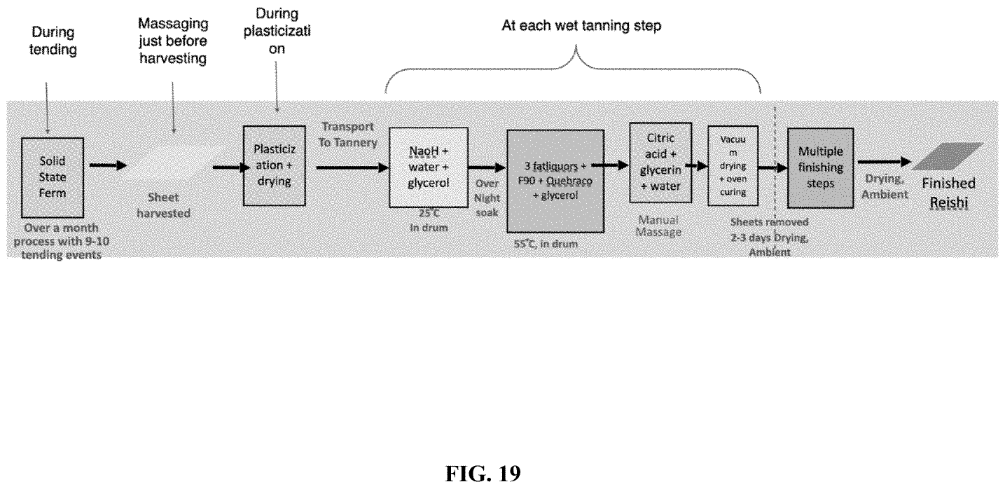

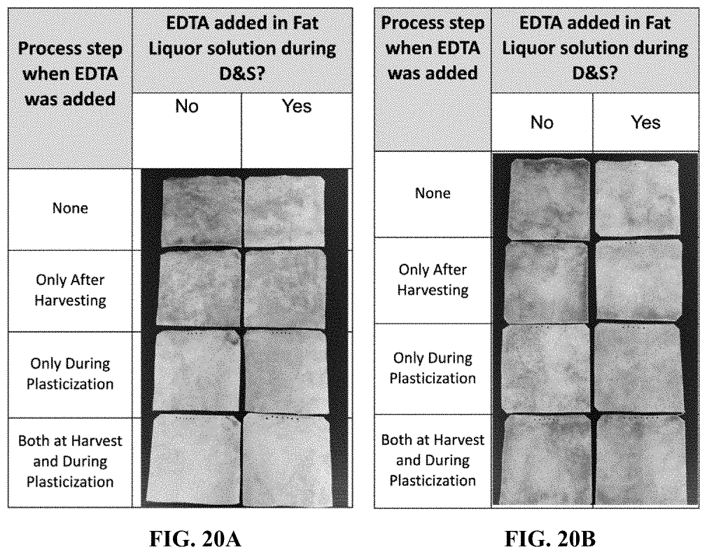

In a preferred embodiment, the above-described treatments are implemented after harvest (last step of harvest/tending), plasticization, and all wet tanning operations as shown in FIG. 20A. FIG. 19 shows a schematic of an exemplar process pipeline 30 (also referred to herein as the “Reishi plasticization and tanning process 30”) including various potential steps wherein EDTA may be applied. For example, EDTA or EGTA may be applied during solid state fermentation (during tending), during sheet harvesting (massaging just before harvesting), during plasticization, and/or at each wet tanning step, as shown in FIG. 19. As shown in FIG. 8, salted forms of EDTA and other chelating agents shown in FIG. 8 may provide a lower impact in terms of browning reduction. In various embodiments, the present inventors have optimized solution conditions to identify ideal concentrations of EDTA, ideal conditions to dissolve in NaOH, and key alternates to EDTA. In some embodiments, each wet step is to include EDTA treatment as described above.

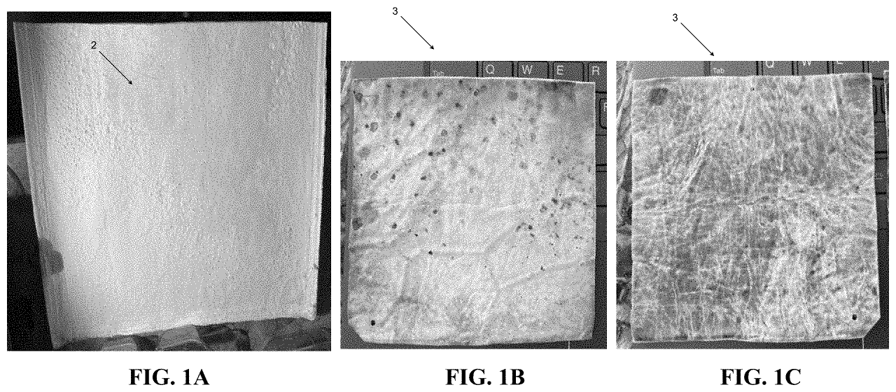

As shown in FIG. 1A-FIG. 1C, in some embodiments the appearance of wet felt sheet before tanning (FIG. 1A) and after tanning (FIG. 1B-1C) may be distinctly different. FIG. 1A shows the front aspect of a wet felt sheet 2 of mycohide prior to tanning. FIG. 1B and FIG. 1C show the front and rear aspects, respectively, of a tanned wet felt sheet (i.e., after tanning) 3. Comparison of the above figures shows that the tanning process results in browning. This enzymatic browning, largely the result of polyphenol oxidases (PPOs), contributes to the darkened color quality of the pictured mycohide sheets.

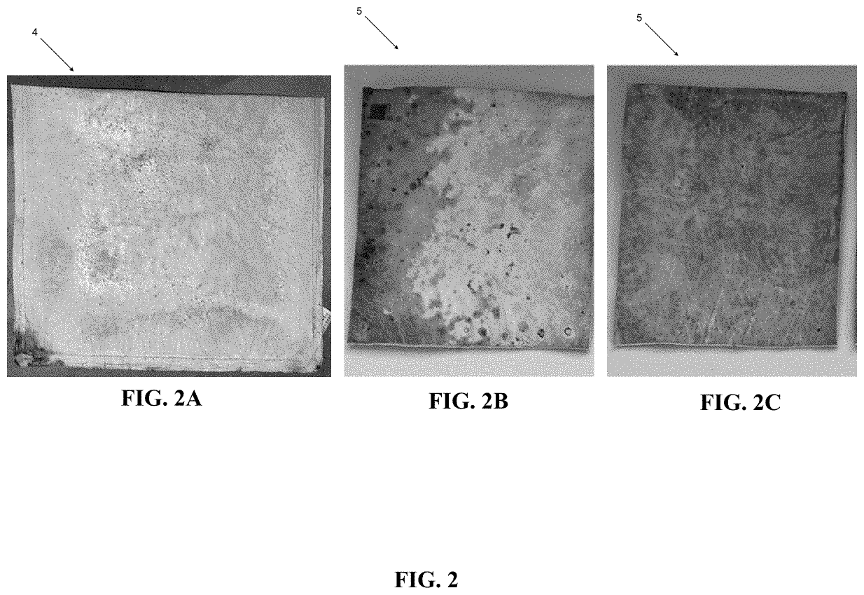

Further to the above, physical and chemical methods are described that inhibit the activity of PPOs and thereby reduce browning in mycohide materials, including those previously subjected to plasticization processes. Relatedly, FIG. 2A shows the front aspect of a pre-tanned plasticized felt sheet 4. In addition, FIG. 2B shows the front aspect of a tanned plasticized felt sheet 5. FIG. 2C shows the back aspect of a plasticized felt sheet after tanning. Similar to the wet felt sheet, the tanning process in plasticized results in browning. This enzymatic browning, largely the result of polyphenol oxidases (PPOs), contributes to the darkened color quality of the pictured plasticized mycohide sheets.

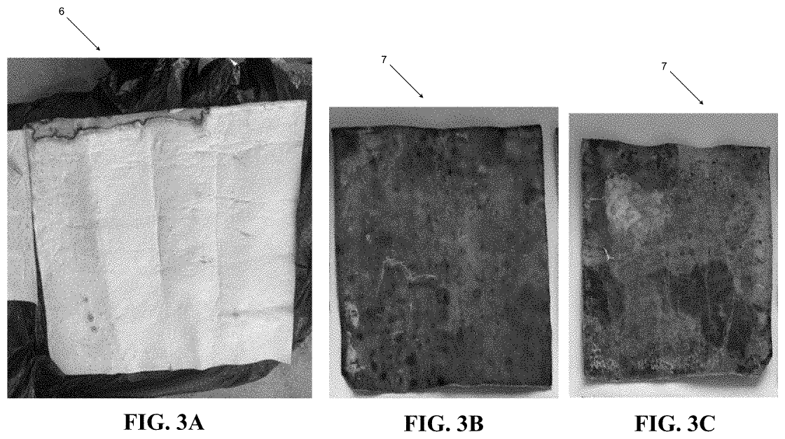

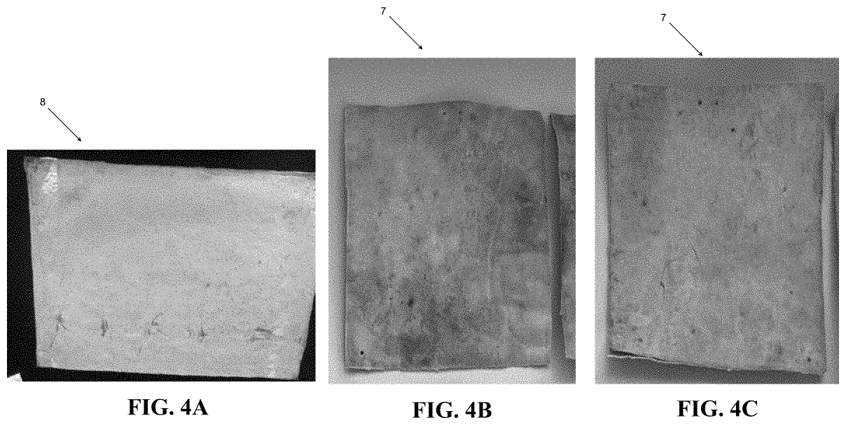

In some embodiments, browning is a prevalent issue in mycohide cotton sheets in addition to felt sheets and plasticized sheets. FIG. 3A shows the front aspect of a pre-tanning wet cotton sheet of mycohide 6. FIG. 3B and FIG. 3C show the front and back aspect, respectively, of the tanned cotton sheet of mycohide 7. FIG. 4A shows the front aspect of a pre-tanned plasticized cotton sheet 8. FIG. 4B and FIG. 4C show the front aspect and back aspect, respectively, of a tanned plasticized cotton sheet 9. Similar to the wet felt and plasticized felt sheets described above, browning emerges as a prevalent issue. Notably, not all sheets brown to the same degree and some sheets do not brown at all in some embodiments. For example, some sheets may be fat-liquored with D&S, and remain very white after a full year.

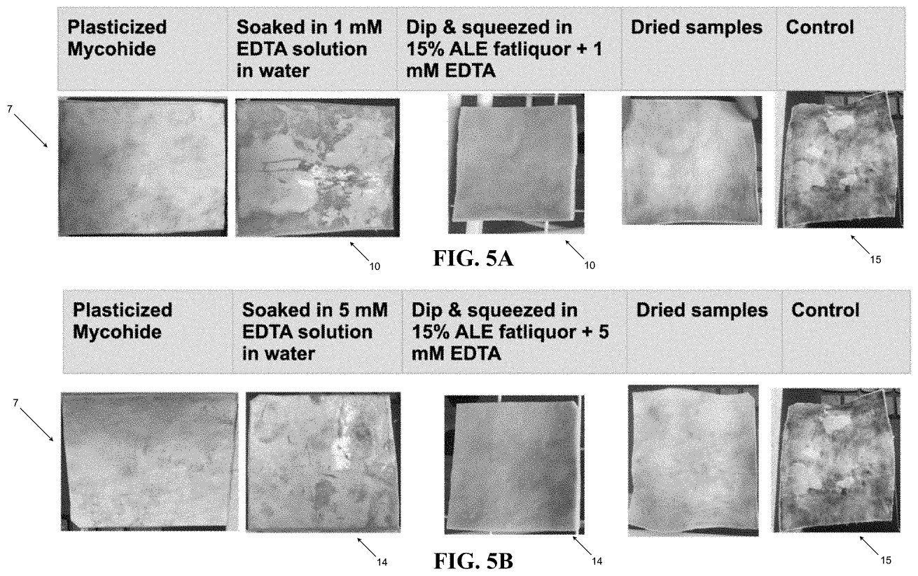

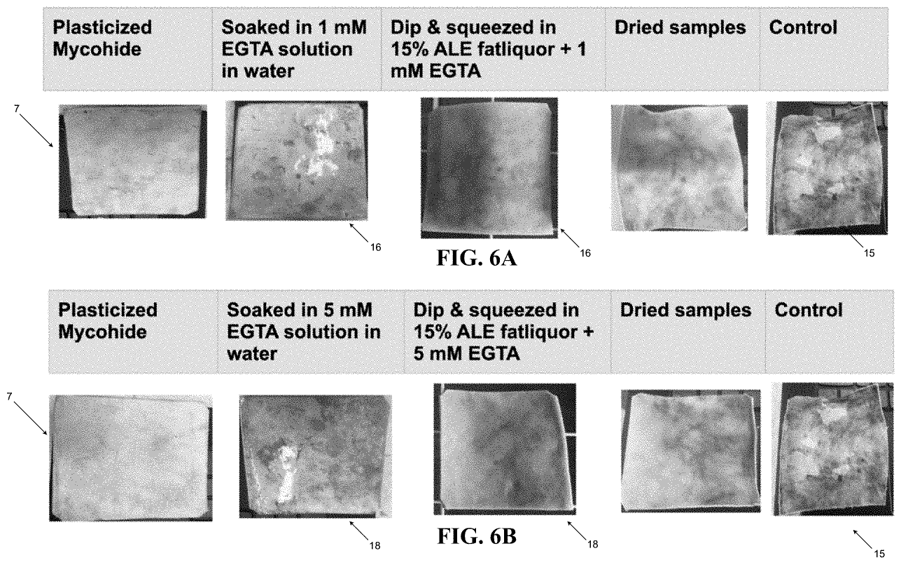

In a preferred embodiment, chelating agents like EDTA and EGTA are used to reduce browning in mycelial biomaterials for various applications including fine mycelium. In some embodiments, EDTA treatment may be beneficially applied at varying concentrations and at any mycelium processing step wherein the mycelium is saturated or moistened. For example, FIG. 5A and FIG. 5B compare the impact of 1 mM EDTA treatment 10 and 5 mM EDTA treatment 14, respectively, against plasticized mycohide 7, dried samples 17, control samples 15, and samples dipped and squeezed in 15% ALE fatliquor+1 mM EDTA. Similarly, FIG. 6A and FIG. 6B compare the impact of 1 mM EGTA 16 and 5 mM EGTA 18, respectively against plasticized mycohide 7, dried samples, control samples 15, and samples dipped and squeezed in 15% ALE fatliquor +1 mM EGTA.

The results from FIGS. 5-6 show that the reduced browning methods described herein (employing either EDTA or EGTA) may be effectively applied to any of the steps in the mycelium processing pipeline wherein the mycelia are in a wet or moist state, including during growth, post-harvest dried or during plasticization, or during downstream and/or tanning steps (i.e., fatliquoring steps). In a preferred embodiment, EDTA is utilized preferentially to EGTA because it achieves greater reductions in browning, as shown in FIGS. 5-6. In other embodiments, a user may choose to mimic the appearance of various dried leathers and hides by utilizing dried samples 17 following the treatment steps shown above, as illustrated in FIG. 6A and FIG. 6B.

In a preferred embodiment, a temperature condition of 50 deg C corresponds with a heat treatment period of 1, 2, or 3 hours. Relatedly, under standard conditions, a temperature condition of 60 deg C corresponds with a heat treatment time period of 1, 2, or 3 hours. In addition, under standard conditions a temperature condition of 70 deg C corresponds with a heat treatment time period of 0.5 hours, 1 hour, or 2 hours maximally. In some embodiments, a temperature condition of 80 deg C corresponds with a heat treatment time period of 0.5 hours, 1 hour, or 2 hours maximally. In other embodiments, a temperature condition of 90 deg C corresponds with a heat treatment time period of 0.5 hours, 1 hour, or 2 hours maximally. Notably, in some embodiments, other chelating agents like diethylenetriaminepentaacetic acid (“DTPA”), GLDA (i.e., Dissolvine GL; glutamic acid tetra sodium salt), and other chelating agents described above may be utilized for chelation activity. In other embodiments, the experiments detailed in FIGS. 5-6 may be repeated with other chelating agents and on sheet sizes up to 10 ft by 10 ft. In some embodiments, a range of heat treatments are also utilized including heating ranges between 50 to 90 deg C.

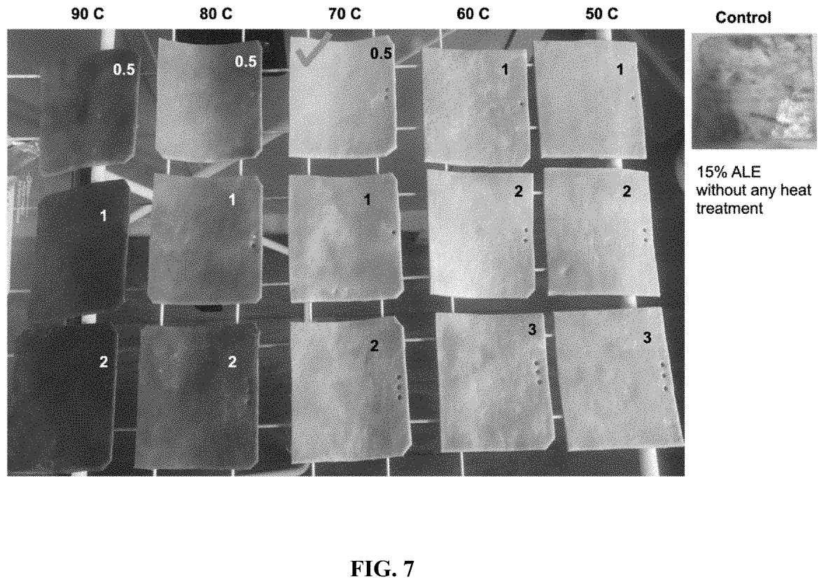

FIG. 7 shows a heat treated (no EDTA) after fatliquoring (wet state) panel 20, with numbering on each mycohide piece representing the time period in hours of heat treatment (heat decreasing from 90 deg C to 50 deg C in intervals of 10 deg C from left to right). A control group prepared with 15% ALE without any treatment is also provided. As shown in FIG. 7, the higher temperature ranges (80-90 deg C) show a darker coloration overall, and that longer heat treatments corresponded with a homogenous somewhat darker coloration. In some embodiments, as shown in FIG. 8, mycelium sheets are heat treated at 50 C, 60 C, 70 C, 80 C, or 90 C (without EDTA treatment) after fatliquoring (dry state before exposing to sunlight). The numbers on each mycelium sheet in FIG. 8 represent the time period in hours for the heat treatment, including 0.5 hour time point labeled on the bottom control sheet. For example, a 90 C treated sheet without EDTA treatment may be heated for 0.5 hours, one hour, or two hours. With increasing time, increased browning is generally visible under these conditions, as shown in FIG. 8. In some embodiments, this heat treated no EDTA (dry state before exposing to sunlight) process 22 provides for an optically heterogenous reduced browning mycelium material.

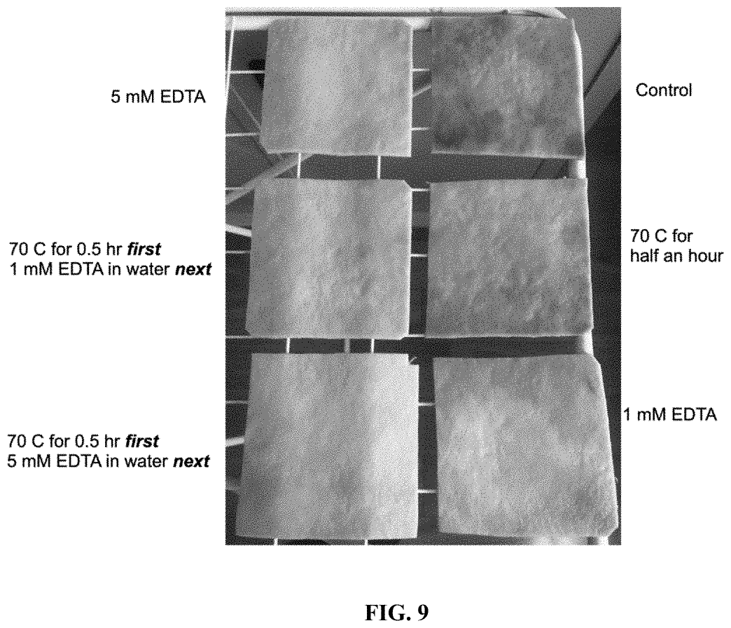

In some embodiments, crusts are treated after fatliquoring (wet state) as shown in FIG. 9. In various embodiments, it is clear that increasing treatment of a crust (also referred to herein as “sheet” or “mycelium sheet”) with increasing levels of EDTA and a 70 C heating step results in increased reduced in browning. For example, the middle two sheets shown in FIG. 9 (“70 C for 0.5 hour first; 1 mM EDTA in water next” and “70 C for half an hour”) show that a 1 mM concentration of EDTA 10 is more effective at reducing browning than a control group 15 (no EDTA). Comparison of these crusts with the bottom row shows that additional EDTA (increasing the concentration from 1 mM EDTA 10 to 5 mM EDTA 14) results in even greater reductions in browning.

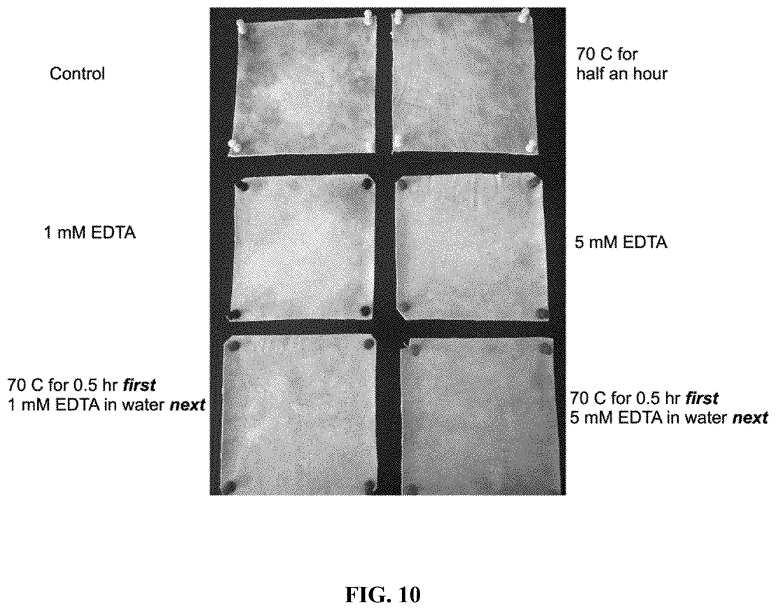

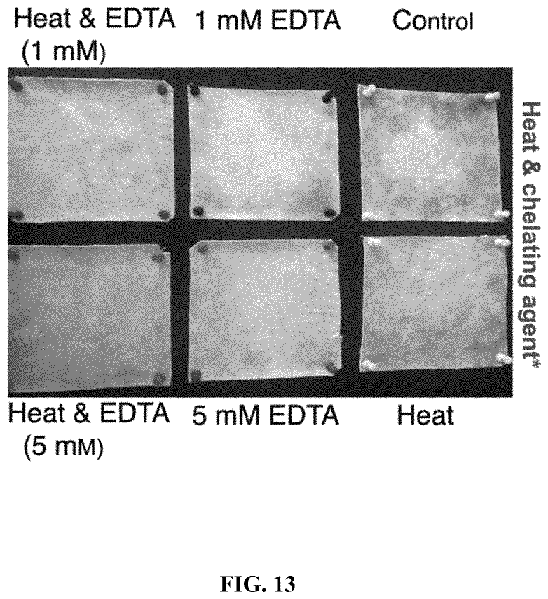

In some embodiments, crusts are treated after fatliquoring in a dry state as shown in FIG. 10. In various embodiments, it is clear that increasing treatment of a dried sheet with increasing levels of EDTA and a 70 C heating step has a lesser impact in terms of browning reduction than the wet state sheets described above. For example, the middle two sheets shown in FIG. 10 (“1 mM EDTA” 10 and “5 mM EDTA” 14) show that a higher concentration of EDTA is not more effective than a lower concentration of EDTA at reducing browning. Inclusion of a 70 C (0.5 hour) heating step first does not change this observation for the dry state experiments of FIG. 10, as exemplified by the bottom sheets. Thus, in a preferred embodiment EDTA treatment can be applied at any wet state in the processing processed for enhanced efficiency in terms of browning reduction. Further to the above, FIG. 13 shows the effect of EDTA application at 1 mM 10 and 5 mM EDTA 14 in the presence and absence of heat.

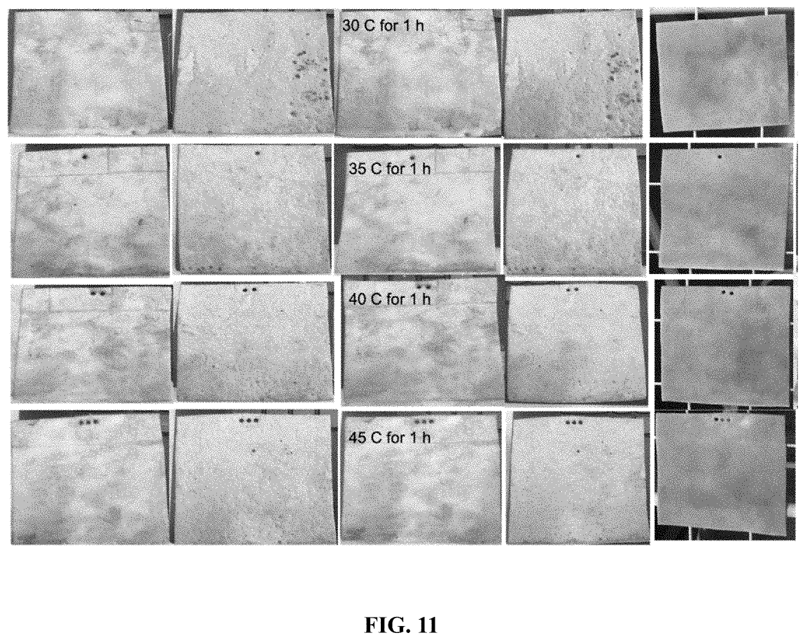

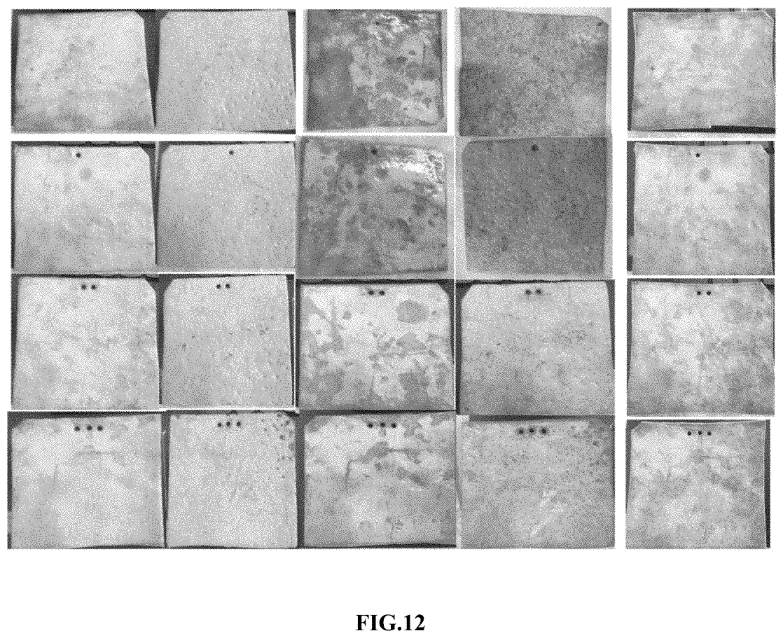

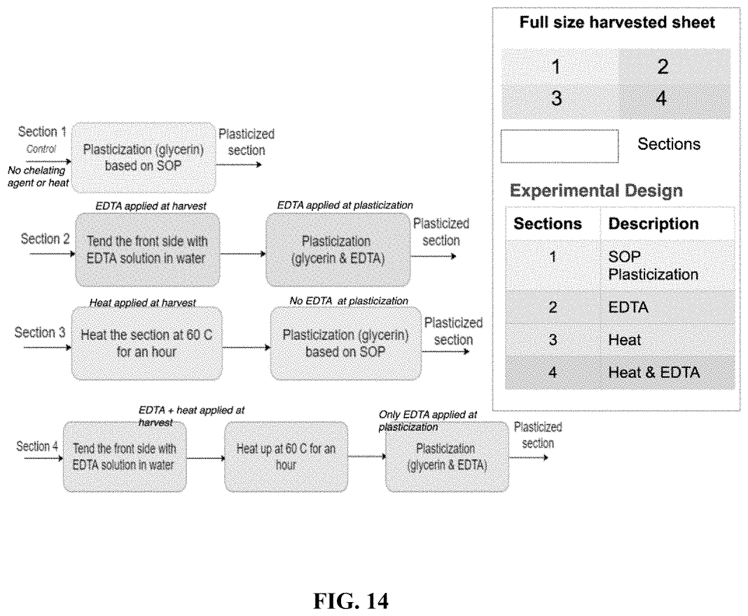

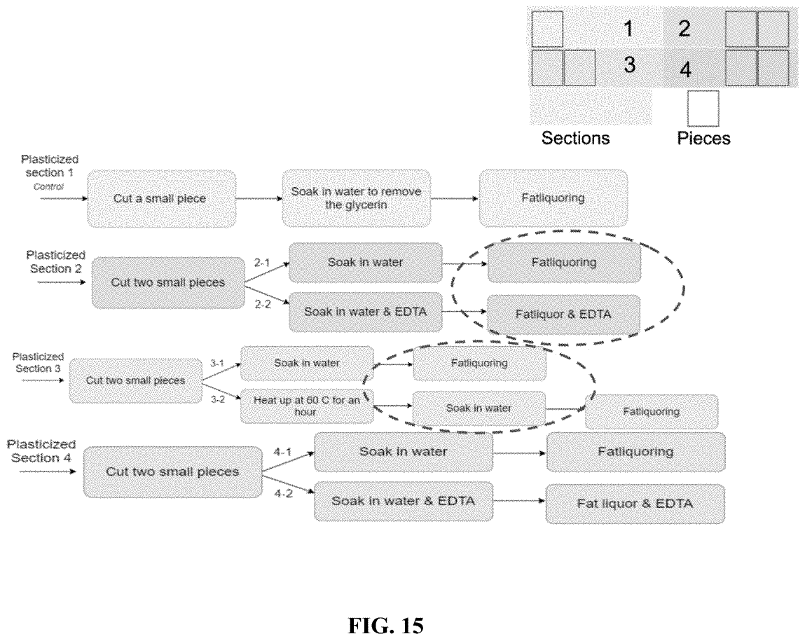

As described above, browning is a prevalent issue in mycohide cotton sheets in addition to felt sheets and plasticized sheets. FIG. 14 shows a schematic of an exemplar SOP plasticization process 24 using glycerin solution on four different harvested mycelium sheets, indicating where EDTA may applied at different points in the SOP plasticization process 24. FIG. 15 shows an exemplar design for a post-plasticization full size harvested mycelium tanning process 26. Relatedly, FIG. 11 and FIG. 12 show a first example of lower temperature ranges 18 and second example of lower temperature ranges 19 on discoloration/browning of plasticized sheets (30 C, 35 C, 40 C, and 45 C temperatures conditions were applied in descending order, resulting in nearly homogenous browning).

In some embodiments, discoloration levels optimized to be EDTA treated are achieved at 30 C, 35 C, 40 C, and 45 C. Implementation of chelating agents and heat treatment strategies discussed above were also applied to both small scale and full size sheets (i.e., contemplated up to 10 ft, by 10 ft). In some embodiments, EDTA solution 14 is added during the tending step in fermentation, plasticization, and/or tanning step. Regarding heat treatment, heat treatment of sheet at the wet stage before plasticization is recommended.

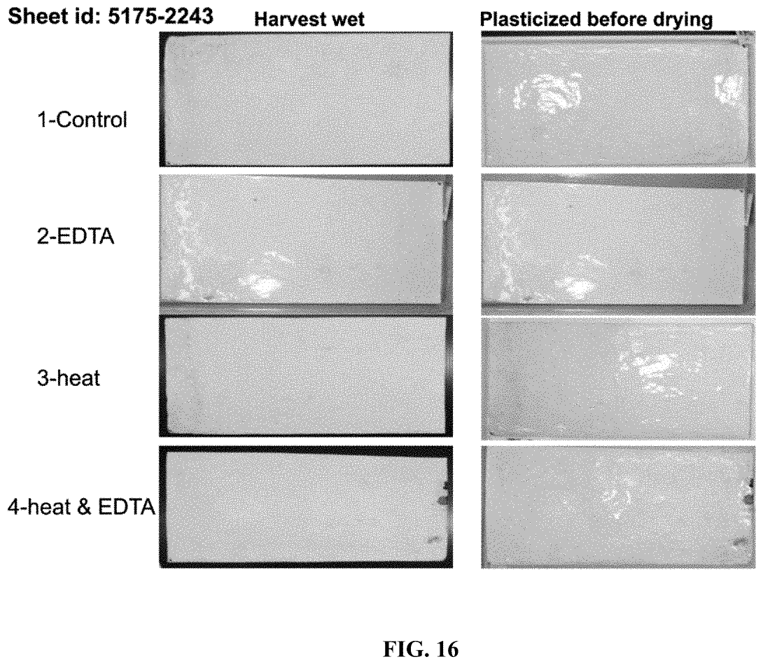

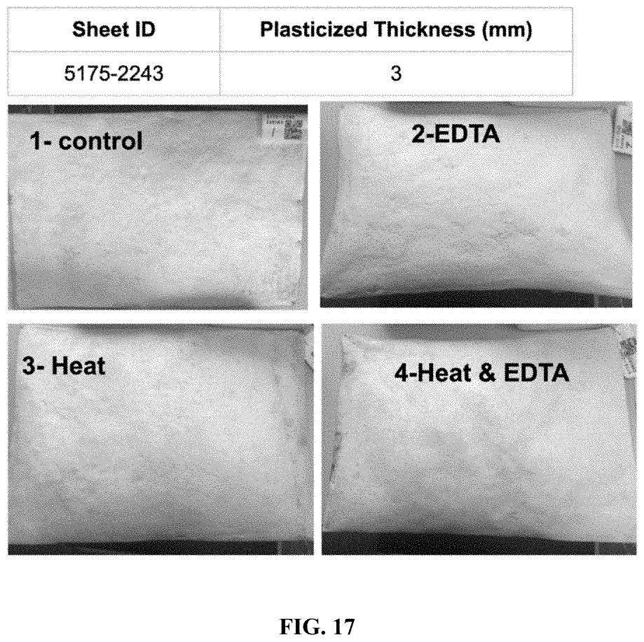

Notably, browning/discoloration of mycohide is irreversible in some embodiments. Ideally, when handling harvested wet or plasticized sheets, browning reducing strategies are employed at the beginning of the plasticization process. FIG. 16 illustrates harvested wet and plasticized sheets in the presence of EDTA, heat, or both EDTA and heat. FIG. 16 shows that the wet sheets were light colored at harvest and didn't discolor substantially after plasticization, including the control without EDTA treatment 14 and/or heat treatment relative to a control 15. In contrast, as shown in FIG. 17, in some embodiments plasticized sheets after drying show some discoloration. This is exemplified in FIG. 17 (Sheet ID 5175-2243) 28, which shows some discoloration and utilizes a plasticized thickness of 3 mm. As described above, FIG. 17 shows dried plasticized sheets after treatment of wet harvested sheets with EDTA 14, heat, or both EDTA and heat, relative to a control sheet 15.

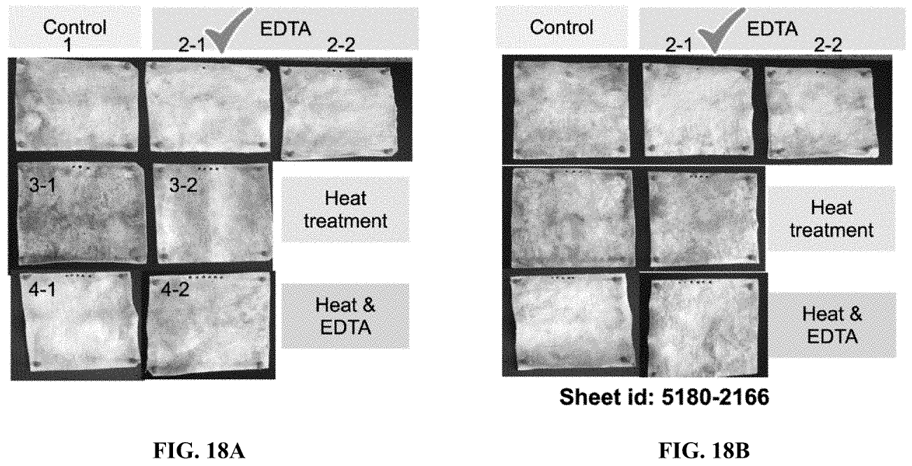

In general, mycelium sheets treated with EDTA have shown the best color uniformity. In contrast, heat treatment with or without EDTA does not show a consistent significant added effect. In some embodiments, it is not necessary to add additional EDTA during tanning (“D&S”) to sheets previously treated with EDTA during harvest or during plasticization. However, in a preferred embodiment, adding EDTA during tanning provided an added whitening effect, as shown in FIG. 18A and FIG. 18B (showing a heat treated no EDTA process 22). As described above, FIG. 18A shows that the application of heat in a first example, with or without EDTA, does not have a consistent significant added effect in terms of browning reduction. Similarly, FIG. 18B shows that the application of heat in a second example (Sheet ID: 5180-2166), with or without EDTA, does not have a consistent significant added effect in terms of browning reduction. In some embodiments, this heat treated no EDTA (dry state before exposing to sunlight) process 22 provides for an optically heterogenous reduced browning mycelium material.

In some embodiments, full sized cotton sheets are treated as defined by four groups: 1) plasticization without any treatment, 2) EDTA treatment only after harvesting sheets (5 mM EDTA 14 in water added on top of mycelia layer after harvest and gently massaged; then plasticize this section in glycerin/water solution), 3) EDTA treatment only during plasticization (plasticize in glycerin/water solution 40%/60% glycerin to water with EDTA at 5 mM), and/or 4) EDTA treatment on both harvested sheets and during plasticization (see above for conditions). Notably, in some embodiments, 5 mM EDTA and 16 mM NaOH are added for any step with EDTA treatment. FIG. 20A and FIG. 20B show the results from a first exemplar and second exemplar set/sheet treatment process applying the conditions described above. As described, EDTA is added at different process steps and under varied fat liquor solution conditions, resulting in an observed decrease in browning. This increased reduction in browning is particularly prevalent in example 1 (FIG. 20A).

### Fungal Materials and Methods

The base inoculum and growth conditions used to produce the reduced browning fungal material (the pre-reduced browning mycelium material) may be varied. The base mycohide is comprised of a fungal inoculum, the fungal inoculum prepared from a desired fungi strain. In some embodiments, the desired fungal strain can include any vegetative, sexual, or asexual structure of a fungus that is capable of growing a new fungal colony. Notably, regardless of the starting materials, subsequent plasticization allows for control of many useful fungal properties, including mechanical properties such as tensile strength, tear strength, abrasion resistance and other chemical properties such as dye fixation.

As described above, the present invention provides a system and methods for reducing browning in a fungal material that was comprised predominately of browned fungal tissues. As described above, referring to FIG. 14, a SOP plasticization process 24 for achieving a reduced browning mycohide is illustrated. After completion of one of the processes exemplified by Section 1-4 shown in FIG. 14, a mycohide sheet may be altern alternatively heated, treated with glycerin and EDTA, and treated with a solvated chelating agent. The resultant optical density of the reduced browning materials, tensile strength, and other characteristics can be optimized at various steps of the SOP plasticization process 24 and other processes shown in FIG. 14, FIG. 15, and/or FIG. 19. In other embodiments, users may mimic the appearance of various leathers and hides in a temporally and spatially controlled manner by optimizing the EDTA soaking and plasticization steps shown in SOP plasticization process 24.

As depicted in FIG. 14, FIG. 15, and/or FIG. 19, methods of making the reduced browning mycohide 10 (or “EDTA-treated sheet”) are widely varied, yet fundamentally include swatches of fresh mycohide transmuted into reduced browning mycohide 10 by way of introducing chelating agents at “wet” processing steps. Chelating agents may be introduced during solid state fermentation (during tending), sheet harvesting (massaging just before harvesting), plasticization, and/or at each wet tanning step, as shown in the Reishi plasticization and tanning process 30 diagram of FIG. 19. This Reishi plasticization and tanning process 30, in which heat treated plasticization occurs through nine steps (with EDTA optionally introduced at several wet steps), provides for an optically homogenous reduced browning mycelium material. As further shown in FIG. 20A and FIG. 20B, EDTA treatment 14 may occur at different process steps and under varied fat liquor solution conditions, resulting in an observed decrease in browning. This increased reduction in browning is particularly prevalent in example 1 (FIG. 20A). At a microscopic scale, this reduced browning process may imbue a variety of chemical rearrangements into the fungal materials, producing a more optically homogeneous product.

As described above, the present invention provides a system and methods for reducing browning in a fungal material that was originally comprised predominately of fungal tissues. The origins of this initial fungal material comprising the pre-reduced browning mycelium material may be varied. In the preferred embodiment, this material is propagated from a colonizable substrate that has been inoculated with a chosen fungus. Preferred species include the Ganodermas, the order Polyporales generally, and including all saprobic fungal candidates that derive sustenance from lignin and cellulose-rich sources.

Below is provided an example of pre-reduced browning mycelium growth conditions. First, a fungal inoculum may be introduced into a substrate within an enclosure or prior to being introduced to the enclosure so as to provide an even distribution of fungus throughout. Next, the substrate is left to colonize. An intermediate layer is established on an open surface of the colonized substrate to control the interaction of the forming fungal tissue structure with the substrate. The presence of a uniform intermediate material atop the substrate enables a consistent surface from which the fungal tissues may grow, supporting uniform expansion of the fungal hyphae into the environment, and providing a determined space for manipulation by chemical and physical controls. Live fungal hyphae grow from the substrate and through the intermediate layer. In some instances, the living tissues that extend through the intermediate layer are manipulated to achieve a material having a desired thickness, shape, size and qualities.

Next, the intermediate layer may be delaminated from the nutrient source out of which it has grown to terminate further growth of the material, or the fungal tissue layer may be delaminated from the intermediate layer, which is left in place and optionally reused. The resultant living fungal tissue structures may optionally be fused with other living fungal tissue structures to create two-dimensional and three-dimensional structures. The final fungal tissue may then be subjected to post-growth processing to achieve desired properties for downstream usage.

The fungal substrate precursor material (“pre-reduced browning mycohide”) may be cultivated in either batch or continuous processes and the fungal tissues may be modified and directed during growth in order to achieve uniform characteristics across a surface, or be engineered to take on distinct local qualities through manipulation of growing tissue, or the addition of particles, fibers, meshes, fabrics, and other additives, armatures, and components. Fungal tissue sheets may be processed via cutting or other forming methods to obtain two-dimensional features and reliefs, or individual sheets may be stacked and grown together to form three-dimensional features, or composed with reinforcements or other structural amendments that may be incorporated into a growing tissue.

In some embodiments, reduced browning fungal materials described herein can behave and perform akin to an animal leather, common industrialized animal skin, or the like. This may be achieved based on the unique molecular structure of reduced browning chitin-based fungal materials and their composites. In some embodiments, post-processing of reduced browning fungal materials may be used to modify its structure or chemical composition, thereby conferring physical qualities according to desired applications.

The foregoing description of the preferred embodiment of the present invention has been presented for the purpose of illustration and description. It is not intended to be exhaustive or to limit the invention to the precise form disclosed. Many modifications and variations are possible in light of the above teachings. It is intended that the scope of the present invention is not limited by this detailed description, but by the claims and the equivalents to the claims appended hereto.

What is claimed is:

1. A system for reducing browning in fungal materials utilizing chelating agents and heat applied to mycelium sheets in various wet processing stages.
